(19) United States
(12) Patent Application Publication
(10) Pub. No.: US 2021/0386426 A1
(43) Pub. Date: Dec. 16, 2021

(54) TRANSCATHETER PERCUTANEOUS BALLOON VALVULOTOMY

(71) Applicant: Ashanga Yatawatta, Bullhead City, AZ (US)

(72) Inventor: Ashanga Yatawatta, Bullhead City, AZ (US)

(21) Appl. No.: 17/303,922

(22) Filed: Jun. 10, 2021

Related U.S. Application Data
(60) Provisional application No. 62/705,080, filed on Jun. 10, 2020.

(51) Int. Cl. A61B 17/11; A61F 2/06

(52) U.S. Cl. CPC A61B 17/11; A61B 2017/00783; A61B 2017/1107; A61F 2/064

(57) ABSTRACT

An in-situ non-reversed femoral-popliteal bypass procedure is provided, the procedural method embodies harvesting a portion of a saphenofemoral junction as the anastomosis for said in-situ non-reversed femoral-popliteal bypass, wherein a portion of the common femoral vein is the patch of the proximal end of the anastomosis. The practitioner may intubate the anastomosis through its distal end with an expandable balloon- prior to grafting said proximal end to the femoral artery; and selectively expanding the expandable balloon at or near said proximal end.

### CROSS-REFERENCE TO RELATED APPLICATION

This application claims the benefit of priority of U.S. provisional application No. 62/705,080, filed 10 Jun. 2020, the contents of which are herein incorporated by reference.

### BACKGROUND OF THE INVENTION

The present invention relates to bypass medical procedures and, more particularly, a transcatheter percutaneous balloon valvulotomy (via an endovenous balloon valvuloplasty catheter) for in-situ non-reversed femoral-popliteal bypass under local anesthesia.

Femoral popliteal bypass (FPB) surgical procedures (also known as lower extremity bypass surgery) are used to treat diseased leg arteries above or below the knee. It is used as a medical intervention to salvage limbs that are at risk of amputation and to improve walking ability in people with severe intermittent claudication (leg muscle pain) among other conditions.

Femoral popliteal bypass is a common vascular surgical procedure with better results than stenting. The patients who will need this are high risk patient due to multiple medical problems and therefore cannot tolerate anesthesia or suffer complications due to anesthesia. In the past, patients who are not candidates for general anesthesia might end up with amputation or death.

Technical factors lead to poor outcomes from utilizing stents and prosthetic grafting; as a result, the vein is the most suitable conduit. Vein harvesting, however, adds more complications and the need for general anesthesia. Specifically, risk of anesthesia and complications related to the long incision for vein harvesting have negative impacts on what would otherwise be a positive outcome compared with stenting or use of prosthetic grafts. In sum, femoral popliteal bypass surgery carries risk with general anesthesia adding more risk.

In short, FPB is limb saving but carries high risk due to the need for general anesthesia and large incisions. Existing femoral popliteal bypass surgical methods encounter problems with vein size mismatch because they use the whole length of the saphenous vein to be opened, which necessitates the large incision and need for general anesthesia as well as invites additional complications.

As can be seen, there is a need for a minimally invasive percutaneous valvulotomy procedural method which can be performed under local anesthesia with the use of a balloon valvuloplasty catheter, enabling better size-matching of artery to saphenous vein, leading to long term patency.

The method embodied in the present invention employs two small incisions at the groin and knee, exposing the common femoral artery and saphenous vein. The practitioner may harvest a section of the saphenofemoral junction from the patient, closing any branching vessel openings. The section of the saphenofemoral junction may include portions of the common femoral vein and the great saphenous vein and the anterior accessory saphenous vein. The practitioner may prepare the proximal and distal ends of the harvested section to be attached in a “proximal anastomosis” and a “distal anastomosis” bypassing a section of one or more other blood vessels, such forming a bypass of the femoral artery and the popliteal artery. The anastomosis may be created using one or more sutures, staples, rings, clips, sleeves, stents, couplers, sealants, glues, and/or adhesives or the anastomosis may be created using one or more laser welding techniques.

The proximal end of the anastomosis may include a portion or cuff of the common femoral vein, while the distal end may include a portion of the great saphenous vein and/or the anterior accessory saphenous vein.

The saphenous vein is divided with a cuff and anastomosed to the artery and the end of the saphenous vein used to close off the cuff. The balloon catheter in used to inflate the vein and disrupt the vein before the anastomosis is completed at the knee. The tributaries of the saphenous vein may be ligated with an endoscopic vein harvest system at the end of the procedure.

Additionally, it can be seen that there is a need for an endovenous balloon valvuloplasty catheter for in-situ non-reversed femoral-popliteal bypass under local anesthesia.

The present invention enables femoral popliteal bypass procedures with small incisions and using a native vein while the patient is under local anesthesia. Previous utilization of non-reversed in-situ saphenous vein have had suboptimal outcomes due to abolishing the valves.

The method embodied in the present invention uses balloon catheter assisted valvuloplasty to improve the outcome with a minimal invasive approach, thereby avoiding long incisions for vein harvesting through providing for two smaller incisions near the groin and knee, both of which can be done under local anesthesia.

### SUMMARY OF THE INVENTION

In one aspect of the present invention, a method for an in-situ non-reversed femoral-popliteal bypass for a patient includes the following: harvesting a section of a saphenofemoral junction of the patient; fashioning the harvested section as an anastomosis for said in-situ non-reversed femoral-popliteal bypass, wherein a proximal end of said anastomosis includes a cuff comprising a portion of the common femoral vein; intubating a distal end of said anastomosis with an expandable device in a collapsed condition; grafting the cuff to a first artery; and selectively expanding the expandable device to an expandable condition adjacent a valve of the anastomosis.

In another aspect of the present invention, the method further includes the urging the expanded expandable device in a retrograde direction; and grafting the distal end of the anastomosis to a second artery, forming a bypass, wherein the first artery is the femoral artery and the second artery is the popliteal artery, wherein the intubating is initiated through a first incision near a knee of the patient, wherein the harvesting is initiated through a second incision near a groin of the patient, wherein the patient is under only local anesthesia, and wherein the distal end of the anastomosis comprises the saphenous vein.

These and other features, aspects and advantages of the present invention will become better understood with reference to the following drawings, description and claims.

### BRIEF DESCRIPTION OF THE DRAWINGS

FIG. 1 is a schematic view of an exemplary embodiment of a procedural step of the present invention;

FIG. 2 is a schematic view of an exemplary embodiment of a procedural step of the present invention;

FIG. 3 is a schematic view of an exemplary embodiment of a procedural step of the present invention;

FIG. 4 is a schematic view of an exemplary embodiment of a procedural step of the present invention;

FIG. 5 is a schematic view of an exemplary embodiment of a procedural step of the present invention;

FIG. 6 is a flowchart of an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a method for an in-situ non-reversed femoral-popliteal bypass for a patient. The procedural method embodies harvesting a portion of a saphenofemoral junction as the anastomosis for said in-situ non-reversed femoral-popliteal bypass, wherein a portion of the common femoral vein is the patch of the proximal end of the anastomosis. The practitioner may intubate the anastomosis through its distal end with an expandable balloon-prior to grafting said proximal end to the femoral artery; and selectively expanding the expandable balloon at or near said proximal end.

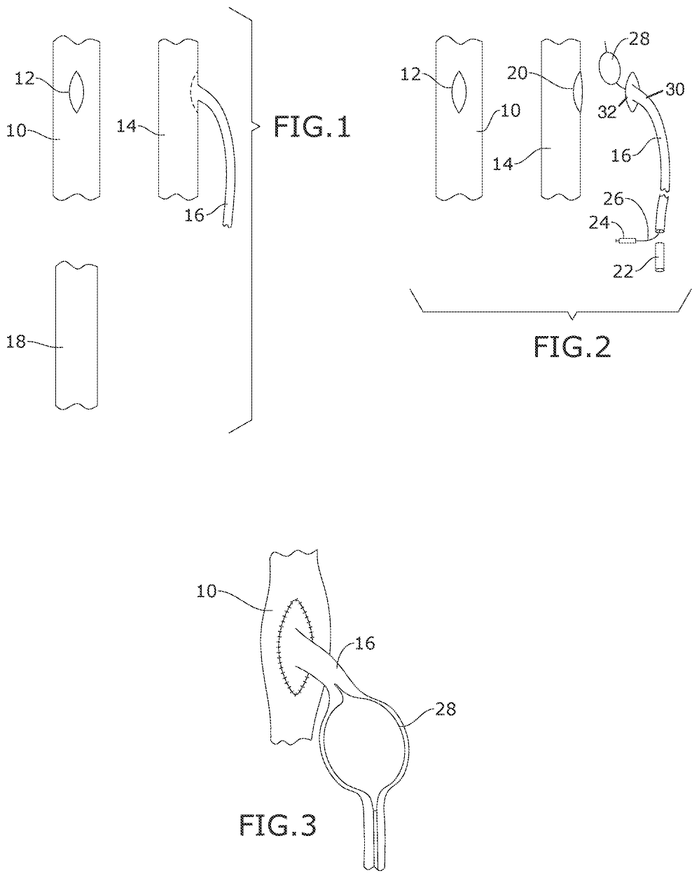

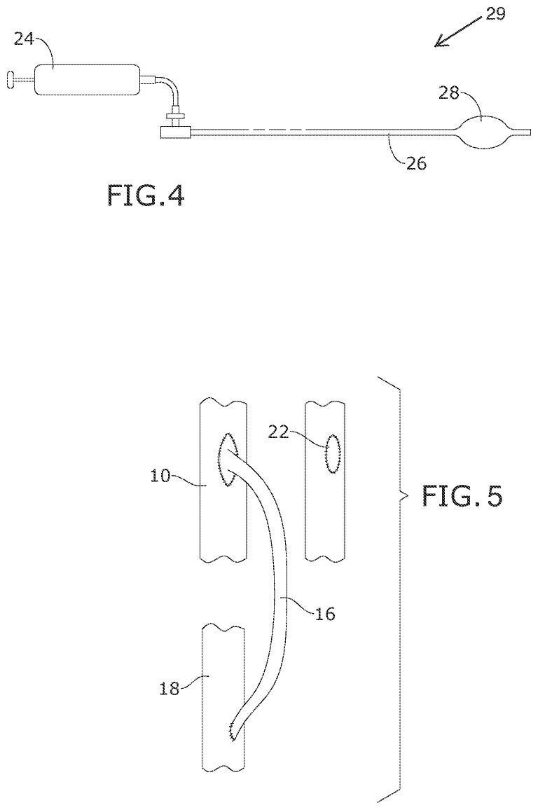

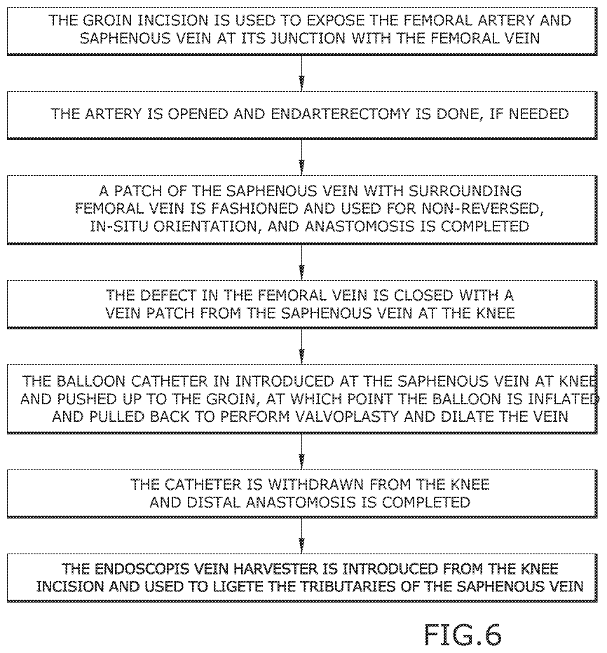

Referring now to FIGS. 1 through 6, the present invention may include a transcatheter percutaneous balloon valvulotomy methodology or procedure embodying a balloon valvuloplasty catheter 29 fluidly connected by a fluid connection 26 to a pressurized fluid source 24, whereby the expandable portion 28. It being understood that the expandable portion 28 may be moved between an expanded condition and a collapsible condition through other means beyond pressurized fluid.

The steps of the transcatheter/transluminal interventional procedure methodology may include the following: first, a groin incision 12 may be used to expose the femoral artery 10 and the saphenous vein 16 at its junction with the common femoral vein 14. The femoral artery 10 may be opened and an endarterectomy done, if needed.

The practitioner may harvest a section 30 of the saphenofemoral junction, (the junction of the saphenous vein 16 with surrounding femoral vein 14) wherein the harvested section 30 is fashioned for non-reversed, in-situ orientation and anastomosis. The practitioner may prepare the proximal and distal ends of the harvested section 30 to be attached in a “proximal anastomosis” and a “distal anastomosis” bypassing a section of one or more other blood vessels. The anastomosis 30 may be used to form a bypass of the femoral artery and the popliteal artery. The anastomosis may be created using one or more sutures, staples, rings, clips, sleeves, stents, couplers, sealants, glues, and/or adhesives or the anastomosis may be created using one or more laser welding techniques.

The proximal end of the anastomosis may include a portion or cuff 32 of the common femoral vein, while the distal end of the anastomosis 30 may include a portion of the saphenous vein 16, including a portion of the great saphenous vein and/or the anterior accessory saphenous vein.

The defect 20 formed in the femoral vein 14 via the removal of the cuff 32 may be closed with a patch 22 from the saphenous vein at or around the knee of the patient. The balloon valvuloplasty catheter 29 may then be introduced into the distal end of the harvested portion 30 (the saphenous vein 16) at or around the knee and pushed up to the groin, at which point, until an end of the balloon valvuloplasty catheter 29 protrudes through the proximal end of the harvested section 30. The proximal end of the anastomosis may be grafted to an arterial vessel 10 over an incision 12 therein. The balloon valvuloplasty catheter 29 may be positioned so the expandable portion 28 is retrograde of the incision 12 adjacent one of the valves of the saphenous vein 16 portion of the harvested section 30. In some embodiments, two to three centimeters retrograde of the incision 12. The expandable portion 28 is selectively expanded based on resistance and visible factors so that the adjacent valve is urged open in the desired direction (in certain embodiments a retrograde direction toward the distal end of the anastomosis). The balloon valvuloplasty catheter 29 may then be pulled back to perform valvuloplasty and dilate the adjacent valve as well as other valves. The balloon valvuloplasty catheter 29 may be withdrawn from the knee and distal anastomosis is completed. An endoscopic vein harvester may be introduced from the knee incision and used to ligate the tributaries of the saphenous vein.

The balloon valvuloplasty catheter 29 or the endovascular venous balloon catheter may be constructed using medical grade plastic with an inflatable balloon at the end. The balloon would be compliant with smooth surface to force open the valves upon pulling back as the blood flow and pressure within the saphenous vein, after the proximal anastomosis, would otherwise keep the valves incompetent. The balloon valvuloplasty catheter 29 may be a systemic unibody device having a balloon 28 tipped endovascular catheter designed for valvotomy.

The balloon valvuloplasty catheter 29 may be used for other types of veins for thrombectomy and valve lysis, for bypass grafts, for the correcting of valve narrowing in the veins, correct valve defects and the like.

The endovascular catheter may be introduced (once the proximal anastomosis is completed) from the distal open end of the vein in such a way that proximal flow is restored. Under ultrasound and fluoroscopy guidance, the balloon may be inflated gently with engagement of each valve while the catheter is pulled back. Each valve may be made incompetent in the process so that flow is further established until the next valve is engaged. The process is repeated until pulsatile flow is noted from the open end, at which point the distal anastomosis can be completed. Tactile feedback from the expandable portion 28 as well as visual feedback from ultrasound and or fluoroscopy may be used to decide the radial and pullback force needed to make the valves incompetent.

Specifically, the proximal anastomosis is completed with use of the proximal end of the saphenous vein at the groin with the optional use of a vein patch 32 fashioned from a portion of the common femoral vein 14. The distal end of the harvested section/anastomosis 30, the saphenous vein portion 16, may be intubated, and the tip of the systemic device slowly introduced under image guidance until the proximal anastomosis. Flow around an arterial occlusion may be restored through the bypass harvested section 30 and the inflated balloon catheter may be slowly withdrawn until resistance if felt, indicating the presence of an engaged valve, which may be confirmed with imaging. Force may be used to perform valvotome while pulling the systemic device and radial force is applied on the balloon. An incompetent valve with pulsatile flow may also be confirmed on imaging. This process is continued until pulsatile flow in noted from the distal open end of the harvested section 30 (saphenous vein 16), at which point, the distal anastomosis is completed.

The method may be modified with a proximal vein harvesting with the second patch from an adjacent vein, in the need for arterial patch angioplasty. In one embodiment, though, the defect 20 on the common femoral vein 10, thus created, will be patched with a second vein patch 22 from the distal end of the saphenous vein 16 at the knee. The tributaries of the newly arterialized saphenous vein may be identified, and a percutaneous vascular stapling device may be used to control them by applying clips with stab incisions. This may be delayed for a week or two, to allow tributaries to mature for better identification. This has the added advantage of minimizing thrombosis of the newly patched common femoral vein at the groin.

In particular, a method of using the present invention may include the following. Under local anesthesia, with preoperative imaging to mark to inflow, runoff and the saphenous vein, the groin incision is made to expose common femoral artery and its branches as well as the common femoral vein and the saphenous vein junction. A distal incision is made in a similar manner to expose the popliteal artery 18 and the saphenous vein 16 at the knee. The proximal artery may be clamped so that inflow is established. If an endarterectomy is needed, the vein harvest method may be modified.

The saphenofemoral junction may be divided and an anastomosis harvested therefrom, wherein the saphenous vein defines the distal end of the anastomosis, and a cuff 32 defining the proximal end of the anastomosis 30, wherein the cuff 32 is a portion of the common femoral vein 14, may be used to construct the proximal anastomosis through grafting the harvested section between the popliteal artery 18 and the femoral artery 10. In other embodiments, other veins with a venous patch may be harvested for use in an endarterectomy, though the indigenous nature of saphenofemoral junction facilitates the minimally invasive (two-incision) nature of procedural method as well as the appropriate match between the arteries and the interconnecting anastomosis.

The endovascular balloon catheter is introduced through the distal end of the vein of the harvested section 30 and negotiated to the proximal anastomosis under imaging. A valvotomy may be done with retrograde forceful pulling back of the catheter with variable radial force on the balloon to induce valvotomy and incompetence. Progressive antegrade pulsatile flow is confirmed with imaging at each valvotomy until the end of the vein is reached. Distal anastomosis is completed. Two weeks later, tributaries of the arterialized saphenous vein are identified on ultrasound and percutaneous venous stapling device is used to close them off, under local anesthesia with stab incisions.

It should be understood, of course, that the foregoing relates to exemplary embodiments of the invention and that modifications may be made without departing from the spirit and scope of the invention as set forth in the following claims.

What is claimed is:

1. A method for an in-situ non-reversed femoral-popliteal bypass for a patient, the method comprising:
harvesting a section of a saphenofemoral junction of the patient;
fashioning the harvested section as an anastomosis for said in-situ non-reversed femoral-popliteal bypass, wherein a proximal end of said anastomosis includes a cuff comprising a portion of the common femoral vein;
intubating a distal end of said anastomosis with an expandable device in a collapsed condition;
grafting the cuff to a first artery; and
selectively expanding the expandable device to an expandable condition adjacent a valve of the anastomosis.

2. The method of claim 1, urging the expanded expandable device in a retrograde direction.

3. The method of claim 2, grafting the distal end of the anastomosis to a second artery, forming a bypass.

4. The method of claim 3, wherein the first artery is the femoral artery and the second artery is the popliteal artery.

5. The method of claim 4, wherein the intubating is initiated through a first incision near a knee of the patient.

6. The method of claim 5, wherein the harvesting is initiated through a second incision near a groin of the patient.

7. The method of claim 6, wherein the patient is under only local anesthesia.

8. The method of claim 7, wherein the distal end of the anastomosis comprises the saphenous vein.
